(19) United States
(12) Patent Application Publication
(10) Pub. No.: US 2019/0351185 A1
(43) Pub. Date: Nov. 21, 2019

(54) Catheter Associated Apparatus and Methods of Making and Using Same

(71) Applicant: Cathetrix Ltd., Jerusalem (IL)

(72) Inventors: Yonatan Assouline, Tel Aviv (IL); Naty Moskovich, Munich (DE); Kfir Beni Elrom, Jerusalem (IL)

(21) Appl. No.: 16/476,554

(22) PCT Filed: Jan. 15, 2018
(86) PCT No.: PCT/IB2018/050231

§ 371 Date: Jul. 9, 2019

Related U.S. Application Data
(60) Provisional application No. 62/446,587, filed on Jan. 16, 2017.
(60) Provisional application No. 62/446,593, filed on Jan. 16, 2017.

(51) Int. Cl. A61M 25/00; A61M 25/10; B33Y 80/00

(52) U.S. Cl. CPC A61M 25/0097; A61M 25/0017; A61M 25/0014; A61M 25/10; B33Y 80/00; A61M 2209/04; A61M 2205/587; A61M 2205/3523; A61M 2205/8206; A61M 2205/18; A61M 2205/581

(57) ABSTRACT

An apparatus comprising: (a) a hub assembly engaged by a podium in a manner which restricts rotation of said hub assembly with respect to said podium to a defined angle of rotation; (b) a swivel installed within said hub assembly in a manner which permits rotation of said swivel with respect to said hub assembly; and (c) a blade installed on an outer surface of said swivel, a cutting edge of said blade facing an inner wall of said hub assembly.

### RELATED APPLICATIONS

This application claims the benefit under 35 U.S.C. § 119(e) of provisional application U.S. 62/446,587 filed 16 Jan., 2017 by ASSOULINE et al. and entitled “Motion Monitor Patch”;

this application also claims the benefit under 35 U.S.C. § 119(e) of provisional application U.S. 62/446,593 filed 16 Jan., 2017 by ASSOULINE et al. and entitled “Catheter Add-On”;

each of which is fully incorporated herein by reference for all that it contains

### FIELD OF THE INVENTION

Various embodiments of the invention are in the field of accessories for use with catheters.

### BACKGROUND OF THE INVENTION

Catheters are used in a variety of medical contexts from draining of urine from the bladder into a collection receptacle in urine catheters or to administer medication or fluids in PVC. Other catheter types include PVC (peripheral venous catheter), PICC line, NG and Nephrostomy catheters.

A urinary catheter is inserted through the urethra into the bladder. Many commonly used urinary catheters include an inflatable balloon near the end of the catheter residing inside the bladder during use. One common catheter type featuring such a balloon is the Foley catheter. Inflation of the balloon serves to anchor the catheter in place. Balloon catheters are commonly used for a variety of purposes including, but not limited to, post-operative care, incontinence management and measurement of urine output.

Catheters feature two inner lumens. The first wider lumen drains urine from the bladder. The second narrower lumen serves to inflate the balloon after insertion of the catheter as well as for deflation of the balloon prior to removal. Inflation is typically with sterile saline, sterile water or air.

The proximal end of the catheter (which remains outside the body) is often “Y” shaped to separate the two lumens from one another. The proximal end of the first wider lumen is often fitted with a connector for a collection vessel. The proximal end of the second narrower lumen is often fitted with an inflation valve configured to engage a syringe.

Balloon catheters are made from a variety of materials such as, for example, Teflon, Silicon, PU (polyurethane), TPU (Thermoplastic Polyurethane), PTFE (polytetrafluoroethylene), PVC (polyvinyl chloride), thermoplastic polyethylene (polyethylene TPE) or Latex and come in different sizes both by length and by diameter.

In some cases, catheters are coated with silicon, and/or hydrophilic coating and/or antimicrobial coating.

Balloon catheters are typically placed inside the urethra by medical staff (doctors and/or nurses) or para-medical care givers.

Additional catheter configurations feature three lumens.

Each year about 100 million Foley catheters are inserted in the United States and about 5 out of 100 patients attempt to remove their catheter without medical supervision (either intentionally or accidentally).

Modern medicine relies on a wide variety of transcutaneous and dermally mounted apparatus and sensors. Historically, making sure that these apparatus/sensors remain properly positioned has been the responsibility of doctors and/or nurses.

### SUMMARY OF THE INVENTION

A broad aspect of the invention relates mitigating tissue damage and/or bleeding caused by removal of a catheter through the urethra while the balloon is inflated.

One aspect of some embodiments of the invention relates to placement of an “Add-On” apparatus external to the outer wall of the catheter. According to various exemplary embodiments of the invention placement occurs after the catheter is inserted, at the point of use but prior to insertion or prior to arrival at the point of use (e.g. at a manufacturing facility or in a specially designated facility within a treatment center). In some cases a catheter is inserted in one facility (e.g. a hospital) and the apparatus is installed on the catheter in a different facility (e.g. nursing home).

Another aspect of some embodiments of the invention relates to attachment of the catheter add-on to a leg of a patient. In some embodiments attachment is via an adhesive patch. Alternatively or additionally, in some embodiments a post or protrusion is inserted into the add-on to facilitate attachment.

Another aspect of some embodiments of the invention relates to a cutting mechanism including a rotating ring with a blade attached thereto. In some embodiments application of a pulling force along the length of the catheter causes the ring to rotate until the blade contacts and cuts the catheter. Once the catheter is cut, the pulling force is dissipated.

It will be appreciated that the various aspects described above relate to solution of technical problems related to undesired removal of the catheter by manual pulling of the catheter by the patients while the retention balloon is still inflated. The manual pulling may be either intentional or the result of inability to comprehend the need for the catheter (e.g. due to dementia, psychosis, retardation or youth).

Alternatively or additionally, it will be appreciated that the various aspects described above relate to solution of technical problems related to accidental removal of the catheter resulting from clinging to external objects (e.g. bed frame or IV pole) and/or accidental pulling by medical staff or caregivers (e.g. when transferring patient from a bed to a chair).

Yet another aspect of some embodiments of the invention relates to local detection of motion of medical hardware with respect to a patient upon which the medical hardware is installed. In some embodiments the hardware is configured as a tube or conduit (e.g. infusion tubing, nasogastric tube, peripheral venous catheter (PVC) or catheter).

According to still another aspect of some embodiments of the invention, an adhesive patch contains circuitry that provides an output signal when the medical hardware moves. In some embodiments the output signal is digital (i.e. either 0 or 1). In other exemplary embodiments of the invention, the output signal is analog (i.e. has a value between 0 and 1).

In some exemplary embodiments of the invention there is provided an apparatus including: (a) a hub assembly engaged by a podium in a manner which restricts rotation of the hub assembly with respect to the podium to a defined angle of rotation; (b) a swivel installed within the hub assembly in a manner which permits rotation of the swivel with respect to the hub assembly; and (c) a blade installed on an outer surface of the swivel, a cutting edge of the blade facing an inner wall of the hub assembly. In some embodiments the apparatus includes a channel sized to engage and retain a catheter along a portion of its length, the channel disposed between an outer surface of the swivel and the inner wall of the hub assembly. Alternatively or additionally, in some embodiments the apparatus includes protrusions on the outer surface of the swivel. Alternatively or additionally, in some embodiments the apparatus includes an alarm trigger responsive to rotational motion of the swivel with respect to the hub assembly. Alternatively or additionally, in some embodiments the apparatus includes a cover closeable over the swivel and the hub assembly. Alternatively or additionally, in some embodiments the apparatus includes alarm circuitry responsive to the alarm trigger. Alternatively or additionally, in some embodiments the apparatus includes a hinge connecting the cover to the hub assembly. Alternatively or additionally, in some embodiments the apparatus includes a cover covering the swivel. Alternatively or additionally, in some embodiments the defined angle of rotation is in the range of 0° to 180° degrees.

In some exemplary embodiments of the invention there is provided an apparatus including: (a) a hub assembly engaged by a podium in a manner which restricts rotation of the hub assembly with respect to the podium to a defined angle of rotation; (b) a swivel installed within the hub assembly in a manner which permits rotation of the swivel with respect to the hub assembly; and (c) an alarm trigger responsive to rotational motion of the swivel with respect to the hub assembly.

In some embodiments the apparatus includes a channel sized to engage and retain a catheter along a portion of its length, the channel disposed between an outer surface of the swivel and an inner wall of the hub assembly. Alternatively or additionally, in some embodiments the apparatus includes protrusions on an outer surface of the swivel. Alternatively or additionally, in some embodiments the alarm trigger is engaged by the hub assembly in a manner which prevents rotation of the alarm trigger with respect to the hub assembly. Alternatively or additionally, in some embodiments the apparatus includes a cover closeable over the swivel and the hub assembly. Alternatively or additionally, in some embodiments the apparatus includes a blade installed on an outer surface of the swivel, a cutting edge of the blade facing an inner wall of the hub assembly. Alternatively or additionally, in some embodiments the apparatus includes alarm circuitry responsive to the alarm trigger. Alternatively or additionally, in some embodiments the apparatus includes a hinge connecting the cover to the hub assembly. Alternatively or additionally, in some embodiments the apparatus includes a cover covering the swivel. Alternatively or additionally, in some embodiments the defined angle of rotation is in the range of 0° to 180° degrees.

In some exemplary embodiments of the invention there is provided a method including: (a) inserting tubing into a patient;(b) engaging a portion of the tubing in an apparatus with a rotating swivel and a cutting blade mounted thereon; and (c) fixing the apparatus in place so that linear motion of the tubing causes rotation of the swivel and causes the cutting blade to cut the tubing. In some embodiments the tubing is made of latex. Alternatively or additionally, in some embodiments the tubing is made of silicon.

In some exemplary embodiments of the invention there is provided a method including: (a) inserting tubing into a patient; (b) engaging a portion of the tubing in an apparatus with a rotating swivel and an alarm trigger; and (c) fixing the apparatus in place so that linear motion of the tubing causes rotation of the swivel and activation of the alarm trigger. In some embodiments the tubing is made of latex. Alternatively or additionally, in some embodiments the tubing is made of silicon. Alternatively or additionally, in some embodiments the alarm trigger activates an alarm on the apparatus. Alternatively or additionally, in some embodiments the alarm trigger activates an alarm at a remote location.

In some exemplary embodiments of the invention there is provided a method including: (a) fashioning a hub assembly, a podium and a swivel; (b) mounting a blade on an outer surface of the swivel, a cutting edge of the blade facing an inner wall of the hub assembly; and (c) installing the swivel within the hub assembly in a manner which permits rotation of the swivel with respect to the hub assembly and mounting the hub assembly on the podium in a manner which restricts rotation of the hub assembly with respect to the podium to a defined angle of rotation. In some embodiments the fashioning includes at least one process selected from the group consisting of injection molding, co-injection, insert injection and over molding. Alternatively or additionally, in some embodiments the fashioning includes additive manufacturing. Alternatively or additionally, in some embodiments the installing includes using connectors. Alternatively or additionally, in some embodiments the mounting includes at least one process selected from the group consisting of insertion in a grove or slot, riveting and heat welding.

In some exemplary embodiments of the invention there is provided a method including: (a) fashioning a hub assembly, a podium and a swivel; (b) mounting an alarm trigger in a position that insures rotation of the swivel activates an alarm; and (c) installing the swivel within the hub assembly in a manner which permits rotation of the swivel with respect to the hub assembly and mounting the hub assembly on the podium in a manner which restricts rotation of the hub assembly with respect to the podium to a defined angle of rotation. In some embodiments the fashioning includes at least one process selected from the group consisting of injection molding, co-injection, insert injection and over molding. Alternatively or additionally, in some embodiments the fashioning includes additive manufacturing. Alternatively or additionally, in some embodiments the installing includes using connectors. Alternatively or additionally, in some embodiments the mounting includes at least one process selected from the group consisting of insertion in a grove or slot, riveting, screwing, snapping, gluing and heat welding.

It will be appreciated that the various aspects described above relate to solution of technical problems associated with timely detection of medical apparatus failure resulting from unwanted motion of a portion of the apparatus in contact with a patient.

Alternatively or additionally, it will be appreciated that the various aspects described above relate to solution of technical problems related to reducing the workload on medical personnel.

Alternatively or additionally, it will be appreciated that the various aspects described above relate to solution of technical problems related to mitigating injury to patients from inadvertent and/or unsupervised removal of medical apparatus.

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a schematic representation of an apparatus according to various exemplary embodiments of the invention installed on a catheter deployed in a body;

FIG. 2a is a perspective view of an apparatus according to one exemplary embodiment of the invention without a catheter;

FIG. 2b is a top view of an apparatus according to another exemplary embodiment of the invention with a catheter inserted in a first operational state;

FIG. 2c is a top view of the apparatus of FIG. 2b in a second operational state;

FIG. 2d is a top view of the apparatus of FIG. 2b in a third operational state; and

FIG. 3 is an exploded view of an apparatus according to one exemplary embodiment of the invention with a catheter positioned for insertion;

FIG. 4 is a schematic representation of an apparatus according to some embodiments of the invention;

FIG. 5 is a schematic representation of an apparatus according to additional embodiments of the invention.

FIG. 6 is an exploded view of an apparatus according to some exemplary embodiments of the invention;

FIG. 7 is a perspective view of an apparatus according to some exemplary embodiments of the invention mounted on patch with catheter inserted and cover open;

FIG. 8 is a perspective view of an exemplary podium suitable for use as part of an apparatus according to some exemplary embodiments of the invention;

FIG. 9 is a perspective view of an exemplary hub assembly suitable for use as part of an apparatus according to some exemplary embodiments of the invention;

FIG. 10a is a perspective view of an exemplary swivel suitable for use as part of an apparatus according to some exemplary embodiments of the invention;

FIG. 10b is a top perspective view of an exemplary swivel cover suitable for use as part of an apparatus according to some exemplary embodiments of the invention;

FIG. 10c is a bottom perspective view of an exemplary swivel cover suitable for use as part of an apparatus according to some exemplary embodiments of the invention;

FIG. 11a is a perspective view of exemplary alarm circuitry suitable for use as part of an apparatus according to some exemplary embodiments of the invention;

FIG. 11b is an exploded view of the circuitry of FIG. 11a;

FIG. 12a is a top view of an apparatus according to an exemplary embodiment of the invention with tubing inserted and cover open in a first operational state;

FIG. 12b is a top view of the apparatus of FIG. 12a with tubing inserted and cover open in a second operational state;

FIG. 12c is a top view of the apparatus of FIG. 12a with tubing inserted and cover open in a third operational state;

FIG. 13 is an exploded view of an apparatus according to some exemplary embodiments of the invention;

FIG. 14a is a perspective view of another exemplary swivel suitable for use as part of an apparatus according to some exemplary embodiments of the invention;

FIG. 14b is a top view of the swivel of FIG. 14a;

FIG. 15a is a perspective view of an exemplary cutting blade suitable for use as part of an apparatus according to some exemplary embodiments of the invention;

FIG. 15b is a perspective view of another exemplary cutting blade suitable for use as part of an apparatus according to some exemplary embodiments of the invention;

FIG. 15c is a perspective view of another exemplary cutting blade suitable for use as part of an apparatus according to some exemplary embodiments of the invention;

FIG. 16 is a simplified flow diagram of a method for using apparatus according to some exemplary embodiments of the invention;

FIG. 17 is a simplified flow diagram of a method for using apparatus according to additional exemplary embodiments of the invention;

FIG. 18 is a simplified flow diagram of a method for manufacturing apparatus according to some exemplary embodiments of the invention; and

FIG. 19 is a simplified flow diagram of a method for manufacturing apparatus according to additional exemplary embodiments of the invention.

### DETAILED DESCRIPTION OF EMBODIMENTS

Embodiments of the invention relate to catheter associated apparatus and methods of making and using such apparatus.

Specifically, some embodiments of the invention are used to cut a catheter, or other tubing, in response to a pulling force applied along the length of the catheter/tubing or by the end points. Alternatively or additionally, some embodiments are used to trigger an alarm in response to a pulling force applied along the length of the catheter or by the end points.

Some embodiments of the invention relate to apparatus for local detection of motion of medical hardware with respect to a patient upon which the hardware is installed.

Specifically, some embodiments of the invention can be used to detect motion of tubing relative to skin.

The principles and operation of various exemplary embodiments of the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Overview

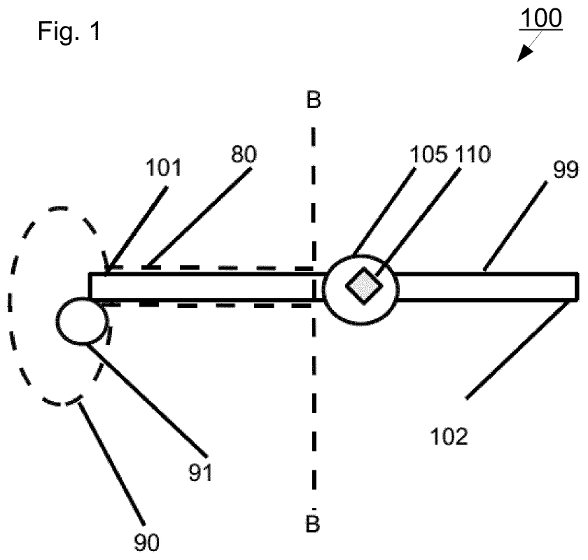

FIG. 1 is a schematic representation of an apparatus indicated generally as 100 according to various exemplary embodiments of the invention installed on a catheter 99, or other tubing, deployed in a body. FIG. 1 depicts an indwelling urinary catheter the invention is amenable to use with all catheter types.

In the drawing distal end 101 of catheter 99 is deployed within urinary bladder 90. A portion of catheter 99 resides within urethra 80 and a portion remains outside the body (i.e. to the right of line B-B). Proximal end 102 of catheter 99 is equipped with a fill port (not depicted) for balloon 91 and a drainage port (not depicted) for urine from bladder 90.

Depicted exemplary apparatus 100 includes a hub assembly 105 adapted to engage and retain catheter 99 therein. Hub assembly 105 is installed sufficiently close to line B-B so that any force applied along the length of catheter 99 is likely to result from engagement of main body 105 or a point on catheter 99 between main body 105 and proximal end 102 of catheter 99. According to various exemplary embodiments of the invention apparatus 100 is installed on catheter 99 so that main body 105 is less than 5, less than 4, less than 3 or less than 2 mm from line B-B. In the depicted exemplary embodiment, apparatus 100 includes a cutting mechanism 110 (and/or alarm mechanism) adapted to sever catheter 99 in response to a pre-defined amount of linear motion of catheter 99 with respect to hub assembly 105. According to various exemplary embodiments of the invention linear motion is caused by patient pulling, and/or by accidental engagement of main body 105, or a portion of catheter 99 between 105 and 102 by an inanimate object (e.g. door knob, bed rail or IV pole).

### First Exemplary Embodiment

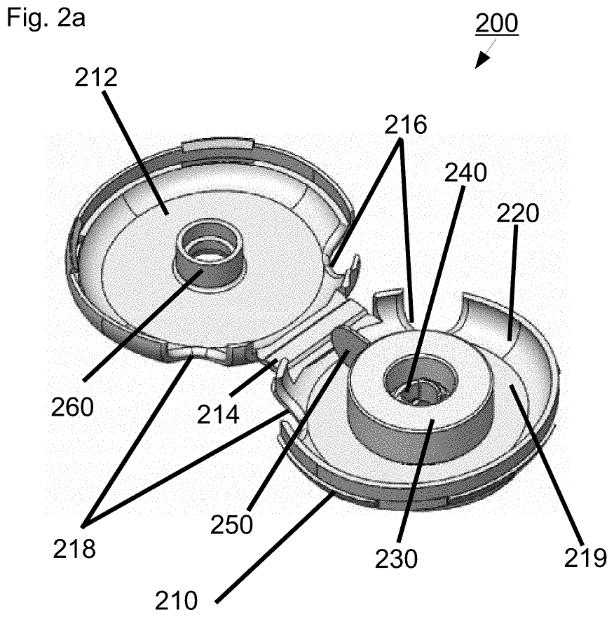

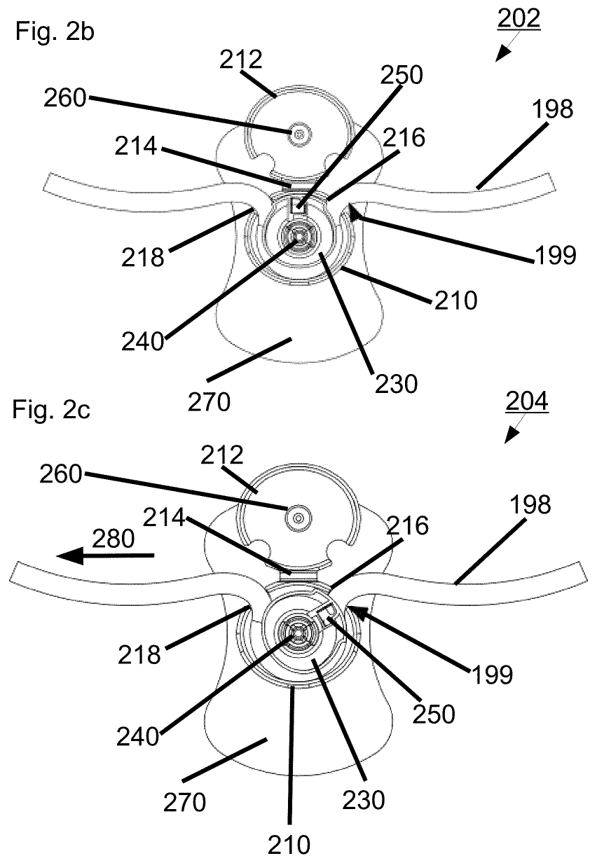

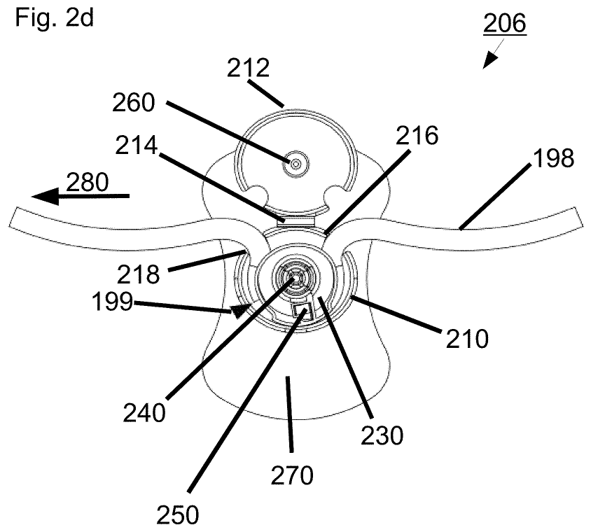

FIG. 2a is a perspective view of an apparatus, in open mode state indicated generally as 200, according to one exemplary embodiment of the invention without a catheter. Depicted apparatus 200 includes a main hub body with halves 210 and 212 joined by a living hinge 214. In use, halves 210 and 212 are rotated with respect to hinge 214 so that top axle portion 260 is aligned with bottom axle portion 240 and apparatus 200 is closed.

When apparatus 200 is closed it defines a channel which accommodates a portion of a catheter length from aperture 216 to aperture 218 via channel 219. In the depicted embodiment, channel 219 is defined by inner wall 220 and rotating cutting mechanism (swivel) 230. Mechanism 230 is free to rotate about axle 240/260. In the depicted embodiment, a single blade 250 is affixed to mechanism 230 and protrudes therefrom. Although blade 250 is fixed with respect to mechanism 230, the blade rotates with respect to axle 240/260 when mechanism 230 rotates.

### Second Exemplary Embodiment

FIGS. 2b, 2c and 2d are top views of an apparatus, indicated generally as 202, 204 and 206 respectively, in a first, second and third operational state respectively. Numbers of parts are as described above for FIG. 2a.

In these figures the apparatus is depicted attached to a stabilizing patch 270. In some embodiments patch 270 is used to affix the apparatus to a leg of a patient.

In these figures a catheter 198 is depicted in channel 219 (see FIG. 1). For clarity, an arbitrary reference point 199 is indicated on catheter 198.

In FIG. 2b, reference point 199 is just outside aperture 216. This is a first operational state in which no pulling force is applied along the length of the catheter. Operational state 202 is “open and ready for catheter installation).

FIG. 2c illustrates a second operational state in which a pulling force 280 is applied along the length of catheter 198. Force 280 causes reference point 199 to pass through aperture 216 into channel 219. As catheter 198 moves, it exerts a force on mechanism 230 which is translated into rotational motion of mechanism 230 which causes blade 250 to move clockwise.

FIG. 2d illustrates a third operational state in which sufficient pulling force 280 has been applied along the length of catheter 198 to cause blade 250 to cut catheter 198. Reference point 199 is now a severed end of catheter 198. In the depicted embodiment, blade 250 begins at a distance from the walls of channel 219. As mechanism 230 rotates, blade 250 approaches and optionally contacts the walls of channel 219. Alternatively or additionally, in some embodiments 210 and 212 are not perfect round shapes but rather flattened a bit to create a cutting board for the blade 250.

### Third Exemplary Embodiment

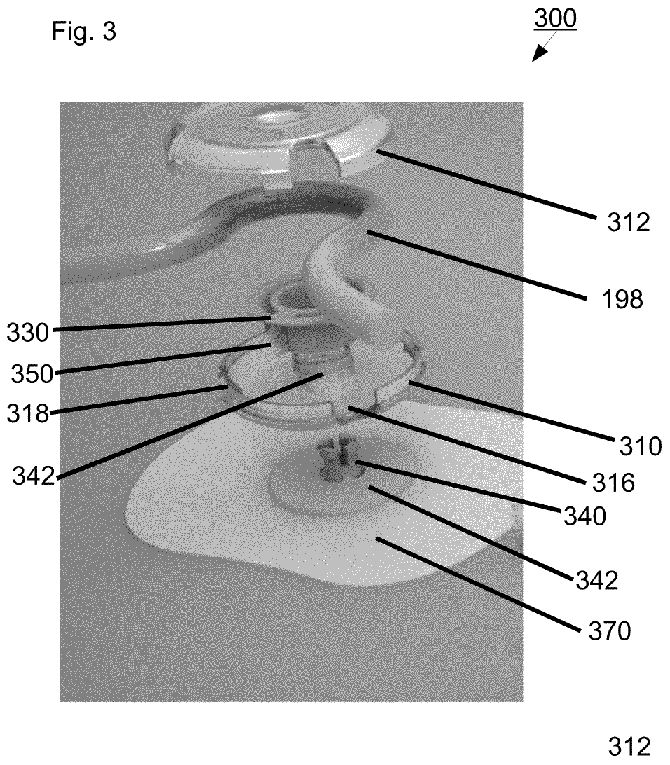

FIG. 3 is an exploded view of an apparatus, indicated generally as 300 according to another exemplary embodiment of the invention with a catheter 198 positioned for insertion. In the depicted embodiment, adhesive patch 370 is provided with a podium 342 and an axle engagement mechanism 340 which fits into axle half 342 of bottom housing half 310 which defines apertures 316 and 318. In the depicted embodiment, rotating cutting mechanism 330 rotates about axle half 342 of bottom housing half 310. As described above, blade 350 is affixed to mechanism 330 and rotates with it. In use, upper housing half 312 is lowered to engage lower housing half 310 and hold catheter 198 in position.

### First Motion Detection Exemplary Embodiment

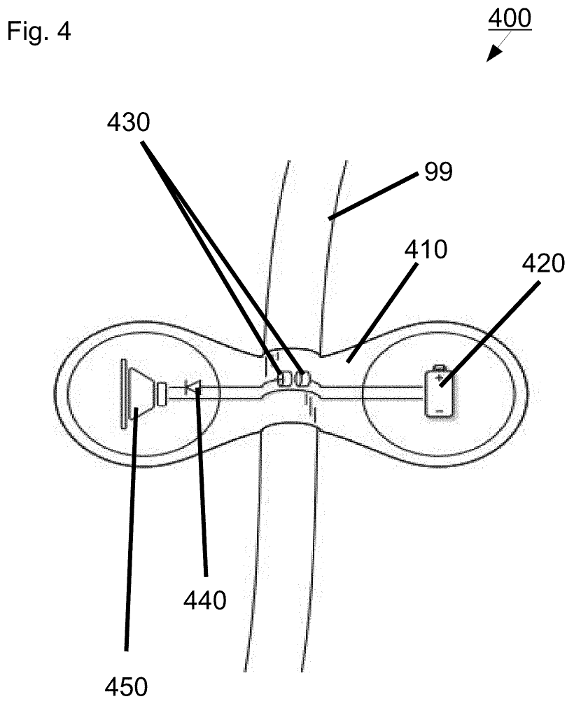

FIG. 4 is a schematic representation of an apparatus, indicated generally as 400 for detection of motion of medical hardware with respect to the body of a patient on which the hardware is installed. In the depicted embodiment, the hardware is tubing 99.

Depicted exemplary apparatus 400 includes an adhesive patch 410 designed and configured to engage both hardware 99 and skin of the patient using the hardware. Integrally formed with, or embedded within, the patch are a power supply 420, detection circuitry 440, a pair of contacts 430 and an output signal generator 450.

The depicted embodiment is digital in the sense that it either provides, or does not provide, a signal depending upon whether contacts 430 are touching one another or not.

In some exemplary embodiments of the invention, detection circuitry 140 is normally open. According to these embodiments, so long as contacts 430 are touching one another, no signal is produced by output signal generator 450. When contacts 430 cease to touch one another, a signal is produced by output signal generator 450.

In other exemplary embodiments of the invention, detection circuitry 440 is normally closed. According to these embodiments, so long as contacts 430 are touching one another, a signal is produced by output signal generator 450. When contacts 430 cease to touch one another, no signal is produced by output signal generator 450.

### Second Motion Detection Exemplary Embodiment

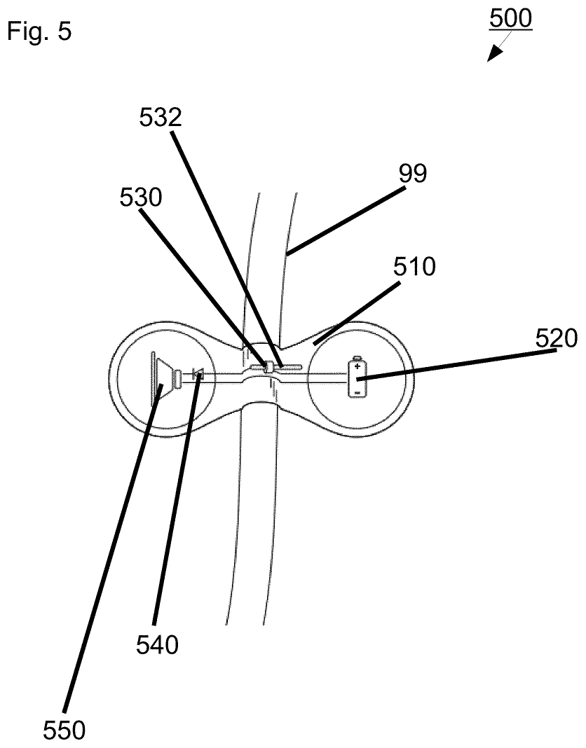

FIG. 5 is a schematic representation of an apparatus, indicated generally as 500 for detection of motion of medical hardware with respect to the body of a patient on which the hardware is installed. In the depicted embodiment, the hardware is tubing 99.

Depicted exemplary apparatus 500 includes an adhesive patch 510 designed and configured to engage both hardware 99 and skin of the patient using the hardware. Integrally formed with or embedded within the patch are a power supply 520, detection circuitry 540, graded rod 532, sensor ring 530 and an output signal generator 550.

The depicted embodiment is analog in the sense that it provides a signal ranging from 0 to 1 (or 1 to 0) depending upon the relative position of ring 530 with respect to rod 532.

In some exemplary embodiments of the invention, detection circuitry 540 is normally open. According to these embodiments, so long as the relative position of ring 530 with respect to rod 532 is “normal”, no signal is produced by output signal generator 550. When the relative position of ring 530 with respect to rod 532 changes, a signal is produced by output signal generator 550. The signal is analog, so its amplitude reflects the magnitude of the change.

In some exemplary embodiments of the invention, detection circuitry 540 is normally closed. According to these embodiments, so long as the relative position of ring 530 with respect to rod 532 is “normal”, a signal is produced by output signal generator 550. When the relative position of ring 530 with respect to rod 532 changes, the signal is produced by output signal generator 550 is dampened. The signal is analog, so the degree of signal dampening reflects the magnitude of the change.

Exemplary Output Signal Generators

Signal generators 450 and 550 are represented iconically as speakers.

In some embodiments the signal produced is a local signal to be observed by the patient. In some embodiments a local audio signal from a buzzer or chime embedded in patch 410 or 510 is heard by the patient. Alternatively or additionally, in some embodiments the signal generator provides haptic feedback to the patient. Alternatively or additionally, in some embodiments the signal generator provides a visual signal to the patient (e.g. blinking of an LED indicator).

Alternatively or additionally, in some embodiments the signal produced is a remote signal to be observed by a caregiver. According to various exemplary embodiments of the invention, remote signals are transmitted via Bluetooth, Wi-Fi, NFC or using an RFID transponder. In some embodiments a wearable device (such as a smart watch) and/or a portable communication device (such as a smartphone) provide supplemental power and increase signal transmission range.

In some embodiments a remote audio signal from a buzzer or chime at a nursing station is heard by medical staff. Alternatively or additionally, in some embodiments the signal generator provides haptic feedback to a staff member via a wearable apparatus. Alternatively or additionally, in some embodiments the signal generator provides a visual signal to the staff (e.g. blinking of an LED indicator on a control console).

### Additional Exemplary Apparatus

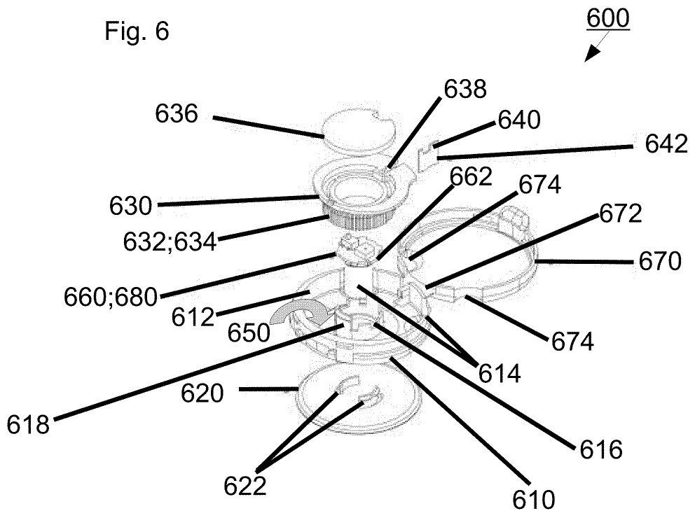

FIG. 6 is an exploded view of an apparatus to cut a catheter (or other medical tubing) in response to a pulling force along the length of the catheter according to some exemplary embodiments of the invention indicated generally as 600.

Depicted exemplary apparatus 600 includes a hub assembly 610 engaged by a podium 620 in a manner which restricts rotation of hub assembly 610 with respect to podium 620 to a defined angle of rotation. In the depicted embodiment, hub connectors 622 on podium 620 restrict rotation.

According to various exemplary embodiments of the invention the defined angle of rotation is in the range of X° to Y°. According to various exemplary embodiments of the invention X° is 0°, 5°, 10°, 15°, 20°, 30°, 35°, 40° or 45° or intermediate numbers of degrees. Alternatively or additionally, according to various exemplary embodiments of the invention Y° is 45°, 60°, 70°, 80°, 90°, 100°, 120° or 180° or intermediate numbers of degrees. In some exemplary embodiments of the invention, the defined angle of rotation is in the range of 0° to 180°. In some exemplary embodiments of the invention, the defined angle of rotation is in the range of 20° to 160°. In other exemplary embodiments of the invention, the defined angle of rotation is in the range of 45° to 120°. In other exemplary embodiments of the invention, the defined angle of rotation is in the range of 60° to 90°. According to various exemplary embodiments of the invention the defined angle of rotation contributes to an ability to place of the device either side of a patient in a mirrored position.

In the depicted embodiment, apparatus 600 includes a swivel 630 installed within hub assembly 610 in a manner which permits rotation of swivel 630 with respect to hub assembly 610. Depicted exemplary apparatus 600 also includes a blade 640 installed on an outer surface 632 of swivel 630. In the depicted embodiment, a cutting edge 642 of blade 640 faces an inner wall 612 of hub assembly 610. In some exemplary embodiments of the invention, a locking mechanism (not visible in this view) holds swivel 630 and/or blade 640 in a desired starting orientation (zero state). According to various exemplary embodiments of the invention the locking mechanism includes breakaway pins and/or a bendable stop/wedge. In the depicted embodiment, blade 640 is engaged by blade slot 638. In the depicted embodiment, engagement arms 618 of hub 610 engage swivel 630.

In the depicted embodiment, apparatus 600 includes a channel 650 sized to engage and retain a catheter (or other medical tubing) along a portion of its length. Channel 650 is disposed between outer surface 632 of swivel 630 and inner wall 612 of hub assembly 610. In some exemplary embodiments of the invention, channel 650 is narrower at one point so linear motion of the catheter/tubing causes swivel 630 to rotate. One exemplary way to provide a narrower portion in channel 650 is to install a circular swivel 630 in an elliptical hub 610. Another exemplary way to provide a narrower portion in channel 650 is to install an elliptical swivel 630 in a circular hub 610.

In some exemplary embodiments of the invention, swivel 630 includes protrusions 634 on outer surface 632. Optional protrusions 634 contribute to an increase in friction between outer surface 632 and a catheter moving in channel 650. In some exemplary embodiments of the invention, outer surface 632 swivel 630, is coated with, or constructed from, a high friction material.

In some exemplary embodiments of the invention, apparatus 600 includes an alarm trigger 660 responsive to rotational motion of swivel 630 with respect to hub assembly 610. According to these embodiments, an alarm indicates catheter is about to be cut or has just been cut. In some exemplary embodiments of the invention, apparatus 600 includes alarm circuitry 680 responsive to alarm trigger 660. In some exemplary embodiments of the invention, circuitry 680 includes a power supply, for example a battery. In the depicted embodiment, tabs 662 are engaged by slots 616 to engage circuitry 680 within hub 610. See the section above entitled “exemplary output signal generators” for more details on alarm circuitry.

In the depicted embodiment, apparatus 600 includes a cover 670 closeable over swivel 630 and said hub assembly 610. Depicted apparatus 600 also includes a hinge 672 connecting cover 670 to said hub assembly 610. In the depicted embodiment, use of a “living hinge” insures correct orientation of cover 670 with respect to hub assembly 610.

Alternatively or additionally, in some embodiments apparatus 600 includes a cover 636 covering swivel 630.

Also visible in FIG. 6 are pairs of complementary half apertures 674 and 614 in cover 670 and hub assembly 610 respectively. When cover 670 is closed, these form apertures that accommodate entry and exit of the catheter from channel 650. In some exemplary embodiments of the invention, these apertures function as static pulleys and swivel 630 functions as a dynamic pulley.

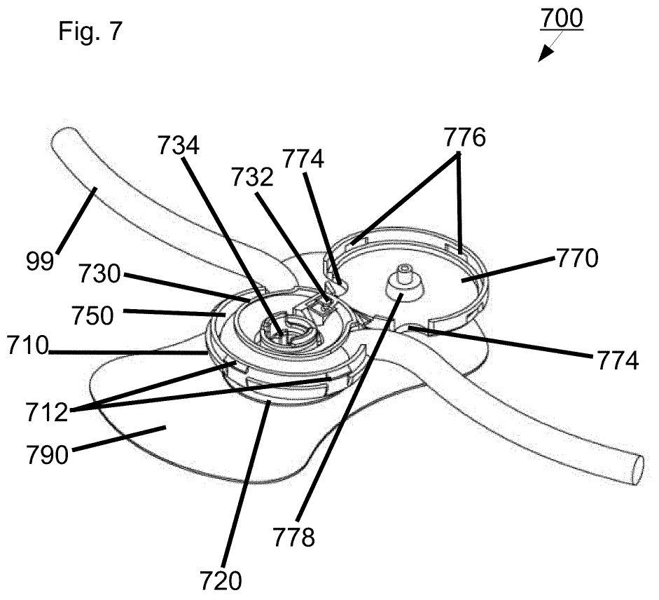

FIG. 7 is a perspective view of an apparatus, indicated generally as 700, according to some exemplary embodiments of the invention mounted on a patch with catheter tubing 99 inserted in channel 750 and cover 770 open. Apparatus 700 is similar to apparatus 600 in terms of function. In depicted apparatus 700, half apertures 774 engage tubing 99 when cover 770 is closed.

When cover 770 is closed slots 776 engage tabs 712 to secure the cover in position. Closing of cover 770 causes pin breaker 778 to break lock pins 734. Breaking of lock pins 734 allows swivel 730 to rotate with respect to hub 710. Rotation of swivel 730 causes the blade (not visible in this view) to move from initial blade position 732 and eventually cut tubing 99.

In some exemplary embodiments of the invention, pad 790 includes an adhesive backing. In some embodiments the adhesive baking is used to affix pad 790 to a skin surface of a patient. In the depicted embodiment, podium 720 is integrally formed with or fixedly attached to pad 790.

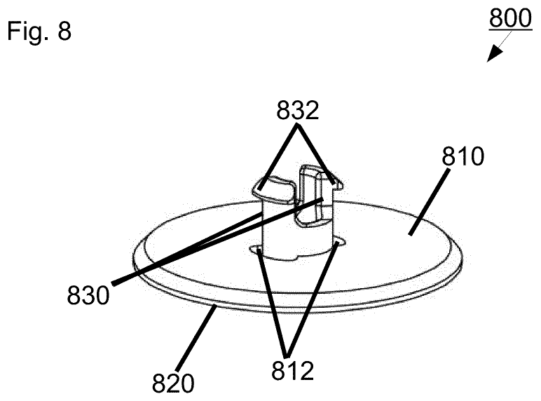

Various components described hereinabove in the context of apparatus 600 and 700 (and hereinbelow in the context of apparatus 1300) are now described in isolation with regard to FIGS. 8 through 11b so that their role within the assembled apparatus is even more clear.

FIG. 8 is a perspective view of an exemplary podium, indicated generally as 800, suitable for use as part of an apparatus according to some exemplary embodiments of the invention. In the depicted embodiment, podium 800 includes a base 810. In some embodiments base 810 is provided with an adhesive backing 820. According to various exemplary embodiments of the invention adhesive backing 820 serves to affix podium 800 to a skin surface of a subject or to a pad (e.g. 7980 in FIG. 7). In the depicted embodiment, slots 812 in base 810 limit a degree of rotation of a hub (not depicted here) mounted on the podium. In the depicted embodiment, upwardly extending arms 830 with engagement hooks 832 allow podium 800 to engage and retain a hub (not depicted).

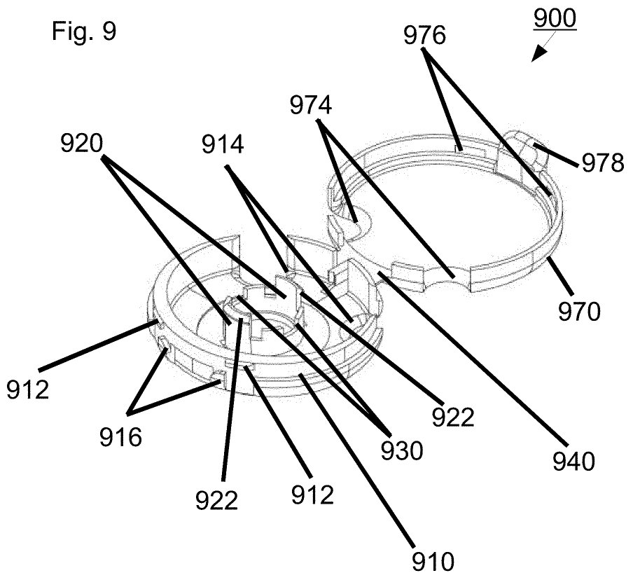

FIG. 9 is a perspective view of an exemplary hub assembly, indicated generally as 900, suitable for use as part of an apparatus according to some exemplary embodiments of the invention.

In the depicted embodiment, hub body 910 is connected to cover 970 by a living hinge 940. When cover 970 covers hub body 910, half apertures 974 align with half apertures 914 to form apertures sized to accommodate relevant catheter tubing. When cover 970 covers hub body 910, engagement slots 976 grasp tabs 912 to hold the cover in a closed position. Alternatively or additionally, when cover 970 covers hub body 910, closure tab 978 mates with engagement hooks 916 to hold the cover in a closed position.

Depicted exemplary hub 900 includes upwardly extending arms 920 terminating in hooks 922 to engage a swivel (not depicted) mounted thereupon.

In the depicted embodiment, additional slots 930 engage hooks 832 (see FIG. 8) of podium arms 830 and/or tabs 662 (see FIG. 6) of an alarm assembly.

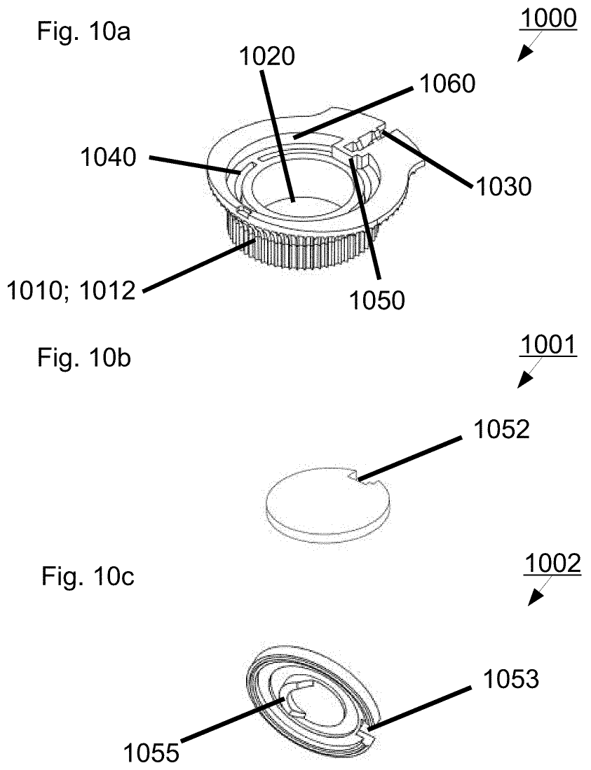

FIG. 10a is a perspective view of an exemplary swivel, indicated generally as 1000, suitable for use as part of an apparatus according to some exemplary embodiments of the invention.

Depicted exemplary swivel 1000 includes inner well 1020. In the depicted embodiment, a slot 1040 for swivel engagement hooks 922 (FIG. 9) is disposed around well 1020. In the depicted embodiment, a well 1060 of a depth sufficient to accommodate a cover is also disposed around well 1020. Optional well 1060 includes a tab 1050 to engage the cover. Depicted exemplary swivel 1000 includes slot 1030 to accommodate a blade (not visible here). In the depicted embodiment, an outer surface 1010 of swivel 1000 is roughened 1012.

FIG. 10b is a top perspective view of an exemplary swivel cover, indicated generally as 1001, suitable for use as part of an apparatus according to some exemplary embodiments of the invention. Depicted exemplary swivel cover 1001 includes a notch 1052 designed and configured to engage tab 1050 (see FIG. 10a) when cover 1001 is seated in well 1060.

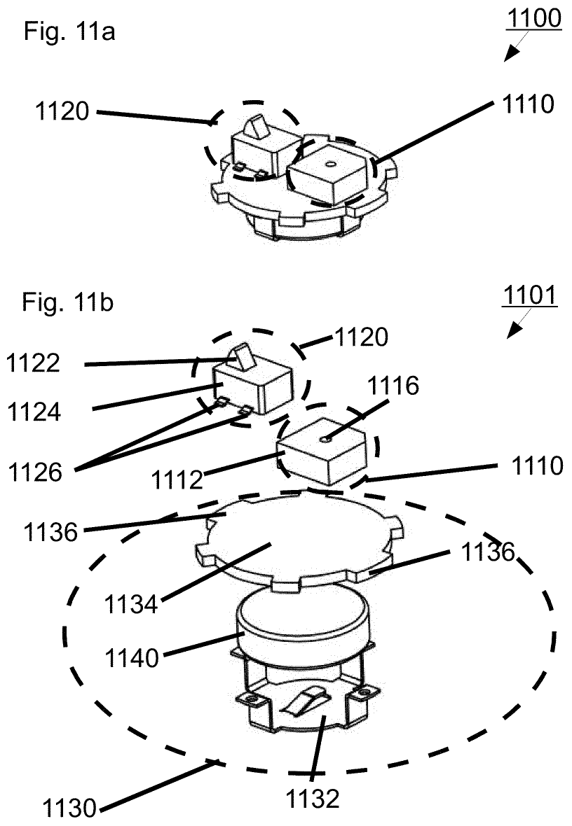

FIG. 10c is a bottom perspective view of an exemplary swivel cover, indicated generally as 1002, suitable for use as part of an apparatus according to some exemplary embodiments of the invention. Depicted exemplary swivel cover 1002 includes a notch 1053 designed and configured to engage tab 1050 (see FIG. 10a) when cover 1001 is seated in well 1060. Depicted exemplary swivel cover 1002 also includes a protrusion 1055 sized and shaped to contact and activate a top mounted tact switch (e.g. 1122 in FIG. 11b) when the swivel rotates relative to the alarm mechanism.

FIG. 11a is a perspective view of exemplary alarm circuitry, indicated generally as 1100, suitable for use as part of an apparatus according to some exemplary embodiments of the invention. The circuitry is depicted in an assembled state. Depicted exemplary circuitry 1100 includes a battery assembly 1130, tact switch with upper activation 1120, and buzzer 1110. According to this exemplary embodiment as the swivel rotates relative to circuitry 1100 (which is fixed with respect to the hub assembly) a protrusion (not visible) on a bottom face of cover 1001 (FIG. 10b) engages and activates tact switch 1120.

One example of a tact switch 1120 suitable for use in some embodiments of the invention is SPVM110200 from Mouser Electronics (Tel Aviv; Israel). One example of a buzzer 1110 suitable for use in some embodiments of the invention is CMT-5023S-SMT-TR (CUI electronics Tualatin, Oreg. USA) available as 102-3743-1-ND from Digi-Key electronics (www.digikey.com). In some exemplary embodiments of the invention, buzzer 1110 provides an output sound of 70 to 90 dB.

FIG. 11b is an exploded view of the circuitry of FIG. 11a indicated generally as 1101.

Depicted exemplary battery assembly 1130 includes a battery retainer 1132 and printed circuit board (PCBA) 1134. In the depicted embodiment, tabs 1136 on PCBA 1134 engage complementary slots in a hub assembly (e.g. 930 in FIG. 9). A battery 1140 is depicted between footplate 1132 and cover 1134. One example of a battery retainer 1132 suitable for use in exemplary embodiments of the invention is BK-335-SM available from www.batteryholders.com (Memory Protection Devices, Inc.; Farmingdale, N.Y.; USA).

Depicted tact switch 1120 includes top mounted switch 1122, housing 1124 and tabs 1126 for attachment to PCBA 1134.

Depicted exemplary buzzer 1110 has a housing 1112 and a hole 1116 through which sound emanates.

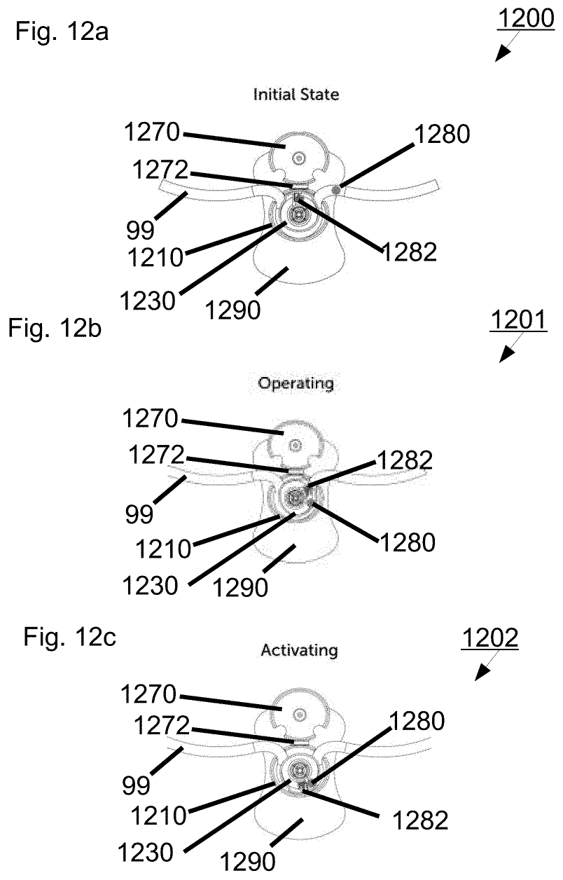

FIGS. 12a,12b and 12c are a series depicting an exemplary apparatus in use in a series of different operational states.

In each figure filled circle [●] 1280 represents a fixed point on tubing 99 and filled triangle [▴] represents a blade position 1282.

In each figure cover 1270 is depicted open with respect to hub 1210 with hinge 1272 intervening. Swivel 1230 holds the blade. Pad 1290 is also visible.

FIG. 12a is a top view, indicated generally as 1200, of an apparatus according to an exemplary embodiment of the invention with tubing 99 inserted and cover 1270 open in a first operational state. The depicted first operational state is reflective of an initial state just after tubing 99 has been installed in the apparatus and before any pulling force has been applied. Filled triangle [▴] 1282 indicates blade position at “twelve o'clock” relative to hub 1210. Filled circle [●] 1280 indicates that the fixed point on tubing 99 is still outside hub 1210.

FIG. 12b is a top view of the apparatus of FIG. 12a, indicated generally as 1201, in a second operational state. The depicted second operational state is reflective of a response to a pulling force on tubing 99. Filled triangle [▴] 1282 indicates blade position at about “two o'clock” relative to hub 1210. Filled circle [●] 1280 indicates that the fixed point on tubing 99 is inside hub 1210 and approaching the blade position.

FIG. 12c is a top view of the apparatus of FIGS. 12a and 12b, indicated generally as 1202, in a third operational state. The depicted third operational state is reflective of a response to a continued pulling force on tubing 99. Filled triangle [▴] 1282 indicates blade position approaching “six o'clock” relative to hub 1210. Filled circle [●] 1280 indicates that the fixed point on tubing 99 is inside hub 1210 and has been bypassed by the blade position. At this stage, the blade has cut tubing 99.

### Further Additional Exemplary Apparatus

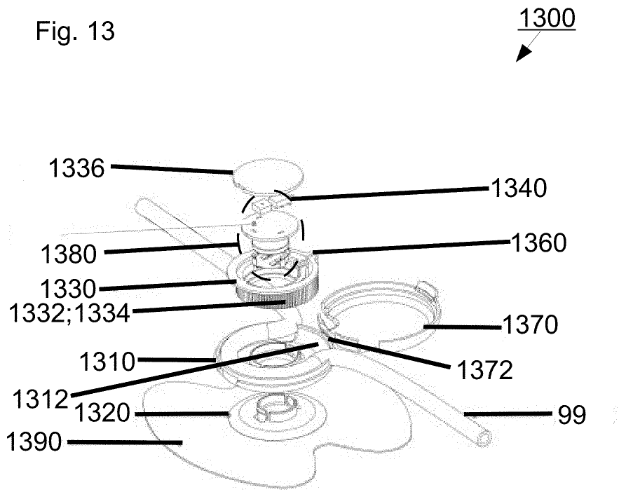

FIG. 13 is an exploded view of an apparatus to detect motion of a catheter (or other medical tubing) in response to a pulling force along the length of the catheter according to some exemplary embodiments of the invention indicated generally as 1300.

Depicted Exemplary apparatus 1300 includes a hub assembly 1310 engaged by a podium 1320 in a manner which restricts rotation of hub assembly 1310 with respect to podium 1320 to a defined angle of rotation. According to various exemplary embodiments of the invention the defined angle of rotation is in the range of X° to Y° degrees. The defined angle of rotation is as set forth hereinabove in detail in the context of FIG. 6.

In the depicted embodiment, podium 1320 is, in turn, mounted on a pad 1390.

Depicted Exemplary apparatus 1300 also includes a swivel 1330 installed within hub assembly 1310 in a manner which permits rotation of swivel 1330 with respect to hub assembly 1310.

In the depicted embodiment, apparatus 1300 includes an alarm trigger 1340 responsive to rotational motion of swivel 1330 with respect to hub assembly 1310. In some exemplary embodiments of the invention, alarm function initiated by trigger 1340 indicates linear motion of catheter as reflected by rotation of swivel 1330. In the depicted embodiment, as swivel 1330 rotates within hub assembly 1310, trigger activator 1360 moves with respect to trigger 1340 and causes trigger activation.

In some exemplary embodiments of the invention, a locking mechanism (not visible in this view) holds swivel 1330 and/or trigger 1340 in a desired starting orientation (zero state). According to various exemplary embodiments of the invention the locking mechanism prevents rotation of the swivel until the apparatus is activated (e.g. by breaking locking pins). In some exemplary embodiments of the invention, activation occurs when a cover is closed.

In the depicted embodiment, apparatus 1300 includes a channel sized to engage and retain a catheter 99 along a portion of its length. In FIG. 13 the channel disposed between an outer surface 1332 of swivel 1330 and an inner wall 1312 of hub assembly 1310 is not visible because it is filled by a portion of catheter tubing 99. In some exemplary embodiments of the invention, the channel is narrower at one point so linear motion of catheter 99 causes swivel 1330 to rotate.

In the depicted embodiment, outer surface 1332 of swivel 1330 includes protrusions 1334. In other exemplary embodiments of the invention, outer surface 1332 is fashioned from, or coated with, a high friction material. This optional feature increases friction with catheter tubing 99.

In the depicted embodiment, alarm trigger 1340 is engaged by hub assembly 1310 in a manner which prevents rotation of alarm trigger 1340 with respect to hub assembly 1310. In the depicted embodiment, engagement is via intervening alarm circuitry 1380.

In the depicted embodiment, apparatus 1300 includes a cover 1370 closeable over swivel 1330 and hub assembly 1310. In the depicted embodiment, cover 1370 is connected to hub assembly 1310 via hinge 1372. In the depicted embodiment, another cover 1336 covers swivel 1330 and alarm circuitry 1380.

In some exemplary embodiments of the invention, apparatus 1300 includes a blade (not depicted) installed on outer surface 1332 swivel 1330 with a cutting edge of the blade facing an inner wall 1312 of hub assembly 1310. In other exemplary embodiments of the invention, 1300 includes a flange (not visible) which retains the catheter and prevents further motion relative to hub assembly 1310.

Depicted Exemplary apparatus 1300 includes alarm circuitry 1380 responsive to alarm trigger 1340. In the depicted embodiment, alarm trigger 1340 is a tact switch with side activation. In some exemplary embodiments of the invention, circuitry 1380 includes a power supply (e.g. battery) and/or a communication port (e.g. USB, Wi-Fi, Bluetooth, RF or Infrared) and/or a visible indicator (e.g. LED or other light) and/or an audible indicator (e.g. buzzer or bell).

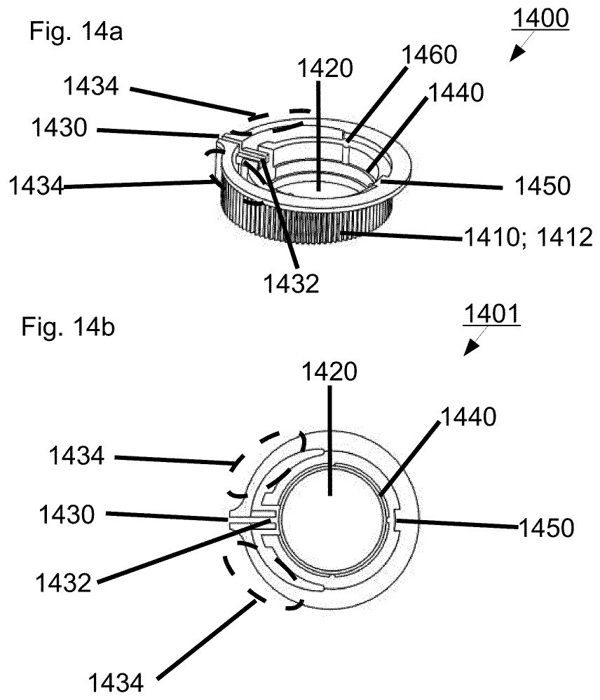

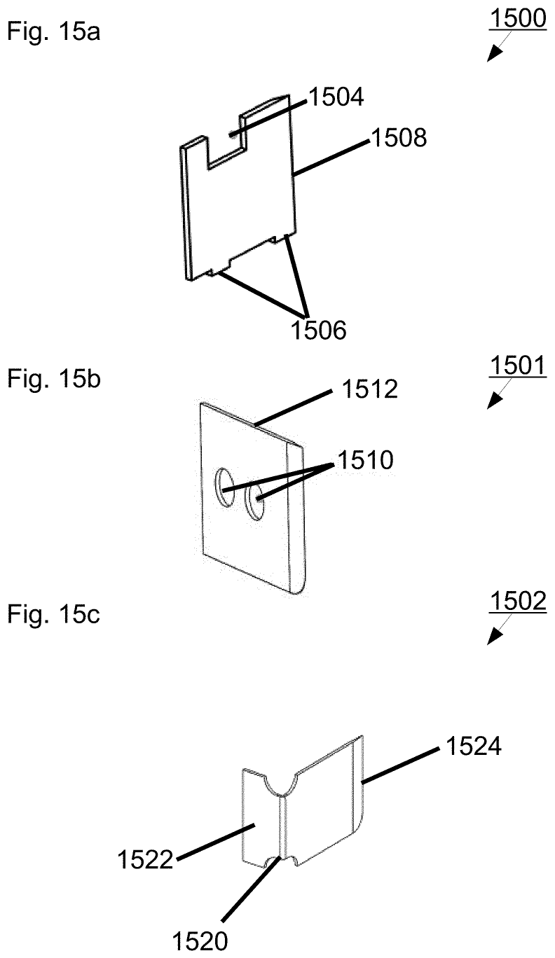

Various components described hereinabove in the context of apparatus 1300 and/or 600 and/or 700 are now described in isolation with regard to FIGS. 14a, 14b, 14a, 15b and 15c so that their role within the assembled apparatus is even more clear.

FIG. 14a is a perspective view of another exemplary swivel, indicated generally as 1400, suitable for use as part of an apparatus according to some exemplary embodiments of the invention. FIG. 14b is a top view of the swivel of FIG. 14a indicated generally as 1401.

Depicted exemplary swivel 1400 has an outer wall 1410 with a roughened surface 1412. An inner well 1420 is surrounded by groove 1440. In some embodiments groove 1440 engages hooks on the swivel (e.g. 922 in FIG. 9).

Depicted exemplary swivel 1400 includes a cover well 1460 and a tab 1450 to engage a matching slot on a cover. In the depicted embodiment, swivel 1400 includes primary trigger activators 1430 at the distal ends of flexible arms 1434 and secondary trigger activators 1432. As swivel 1400 rotates within the hub, primary trigger activators 1430 contact an inner wall of the hub. This contact exerts a force on primary trigger activators 1430 and causes deformation of flexible arms 1434. Deformation of flexible arms 1434 is translated into motion of secondary trigger activators 1432. Motion of secondary trigger activators 1432 operates the trigger (e.g. 1340 in FIG. 13) and sets off an alarm.

FIG. 15a is a perspective view of an exemplary cutting blade suitable for use as part of an apparatus according to some exemplary embodiments of the invention indicated generally as 1500. Exemplary blade 1500 has a slot 1504 and/or tabs 1506 which conform to complementary structures on the swivel so that the blade is held with cutting edge 1508 in the correct orientation.

FIG. 15b is a perspective view of another exemplary cutting blade suitable for use as part of an apparatus according to some exemplary embodiments of the invention indicated generally as 1501.

Exemplary blade 1501 has holes 1510 to facilitate attachment to the swivel. According to various exemplary embodiments of the invention attachment is via rivets and/or screws and/or adhesive and/or heat welding and/or soldering and/or over molding of plastic part of swivel with the blade or by snaps into the holes or grooves at the blade. Attachment serves to hold cutting edge 1512 in the correct orientation.

FIG. 15c is a perspective view of another exemplary cutting blade suitable for use as part of an apparatus according to some exemplary embodiments of the invention indicated generally as 1502.

Exemplary blade 1502 has a flex line 1520. Bending at flex line 1520 forms a retention tab 1522. When retention tab 1522 is inserted in a retention slot on the swivel, cutting edge 1524 is held in the correct orientation.

### Exemplary Method of Cutting Medical Tubing

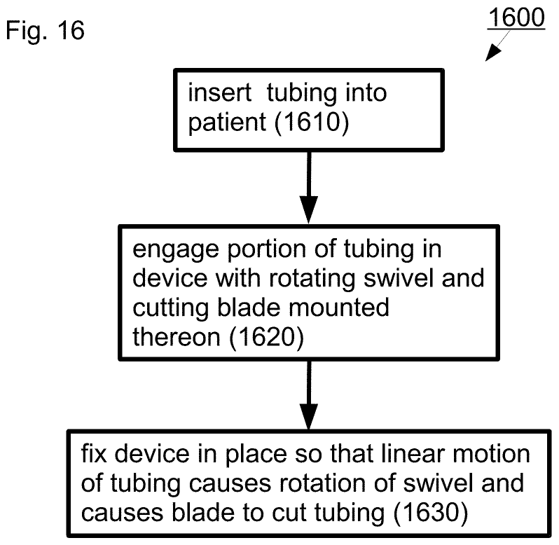

FIG. 16 is a simplified flow diagram of a method, indicated generally as 1600, for using apparatus according to some exemplary embodiments of the invention to cut catheter tubing when it moves with respect to the apparatus.

Depicted exemplary method 1600 includes inserting 1610 tubing into a patient, engaging 1620 a portion of the tubing in an apparatus with a rotating swivel and a cutting blade mounted thereon. In the depicted embodiment, method 1600 includes fixing 1630 the apparatus in place so that linear motion of the tubing causes rotation of the swivel and causes the cutting blade to cut the tubing.

According to various exemplary embodiments of the invention the tubing is made of latex, silicon, polyurethane (PU), polyvinylchloride (PVC), NYLON, TEFLON, TPU (Thermoplastic Polyurethane), PTFE (polytetrafluoroethylene), thermoplastic polyethylene (polyethylene TPE) or PET (polyester; polyethylene terephthalate) or PETG (Poly-Ethylene Terephthalate Glycol).

In some exemplary embodiments of the invention, the swivel rotates within a fixed hub.

### Exemplary Alarm Method

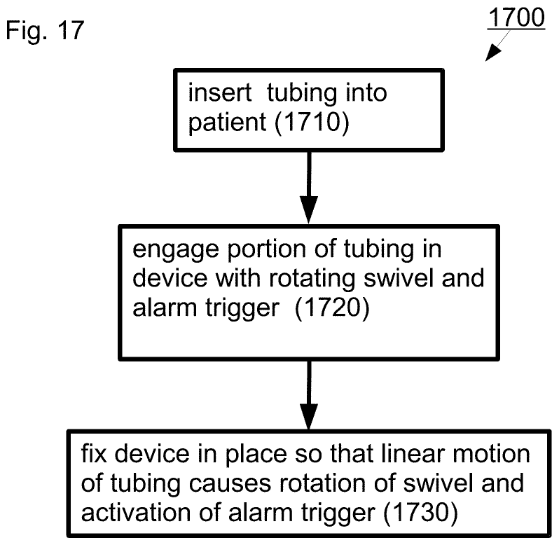

FIG. 17 is a simplified flow diagram of a method, indicated generally as 1700, for using apparatus according to additional exemplary embodiments of the invention to set off an alarm.

Depicted exemplary method 1700 includes inserting 1710 tubing into a patient and engaging 1720 a portion of the tubing in an apparatus with a rotating swivel and an alarm trigger. In the depicted embodiment, method 1700 includes fixing 1730 the apparatus in place so that linear motion of said tubing causes rotation of said swivel and activation of said alarm trigger.

According to various exemplary embodiments of the invention the tubing is made of latex, silicon, polyurethane (PU), polyvinylchloride (PVC), NYLON, TEFLON, TPU (Thermoplastic Polyurethane), PTFE (polytetrafluoroethylene), thermoplastic polyethylene (polyethylene TPE) or PET (polyester; polyethylene terephthalate) or PETG (Poly-Ethylene Terephthalate Glycol).

In some exemplary embodiments of the invention, the alarm trigger activates an alarm on the apparatus. According to various exemplary embodiments of the invention the alarm is audible and/or visible. Alternatively or additionally, in some embodiments the alarm trigger activates an alarm at a remote location. According to various exemplary embodiments of the invention remote activation is via Bluetooth, RF, Wi-Fi infrared signal, NFC or wired connection. In some exemplary embodiments of the invention, the signal is relayed via one or more smart devices (e.g. phone or watch). Alternatively or additionally, in some embodiments the signal is transmitted to a nursing station and/or a smart device (e.g. belonging to medical personnel or caregiver).

In some exemplary embodiments of the invention, the swivel rotates within a fixed hub.

### First Exemplary Production Method

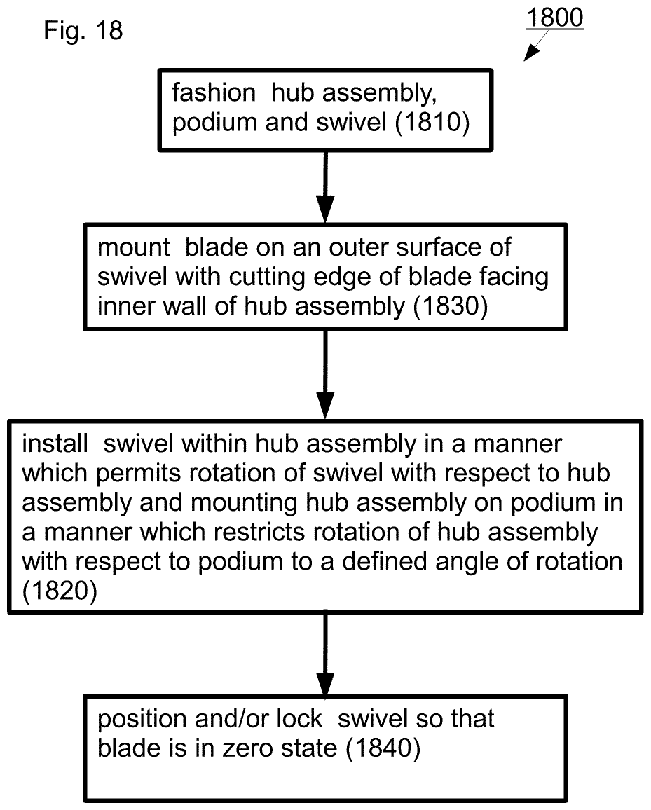

FIG. 18 is a simplified flow diagram of a method for manufacturing and/or assembling apparatus according to some exemplary embodiments of the invention indicated generally as 1800. Depicted exemplary method 1800 includes fashioning 1810 a hub assembly, a podium and a swivel and installing 1820 the swivel within the hub assembly in a manner which permits rotation of the swivel with respect to said hub assembly and mounting the hub assembly on the podium in a manner which restricts rotation of the hub assembly with respect to the podium to a defined angle of rotation. In the depicted embodiment, method 1800 includes mounting 1830 a blade on an outer surface of said swivel, a cutting edge of said blade facing an inner wall of said hub assembly. In some embodiments method 1800 includes positioning and/or locking 1840 the swivel so that the blade is in a desired starting orientation (zero state).

According to various exemplary embodiments of the invention fashioning 1810 includes injection molding and/or co-injection and/or insert injection and/or over molding. Alternatively or additionally, in some embodiments fashioning 1810 includes additive manufacturing.

In some exemplary embodiments of the invention installing 1820 includes using connectors (e.g. screws, snaps, rivets, adhesive or glue).

In some embodiments mounting 1830 includes at least one process selected from the group consisting of insertion in a grove or slot, riveting and heat welding. In some exemplary embodiments of the invention, rivets are plastic pegs or bumps expanded by melting.

In some embodiments mounting 1830 (of the blade on the swivel) occurs prior to installing 1820 (swivel within the hub).

### Second Exemplary Production Method

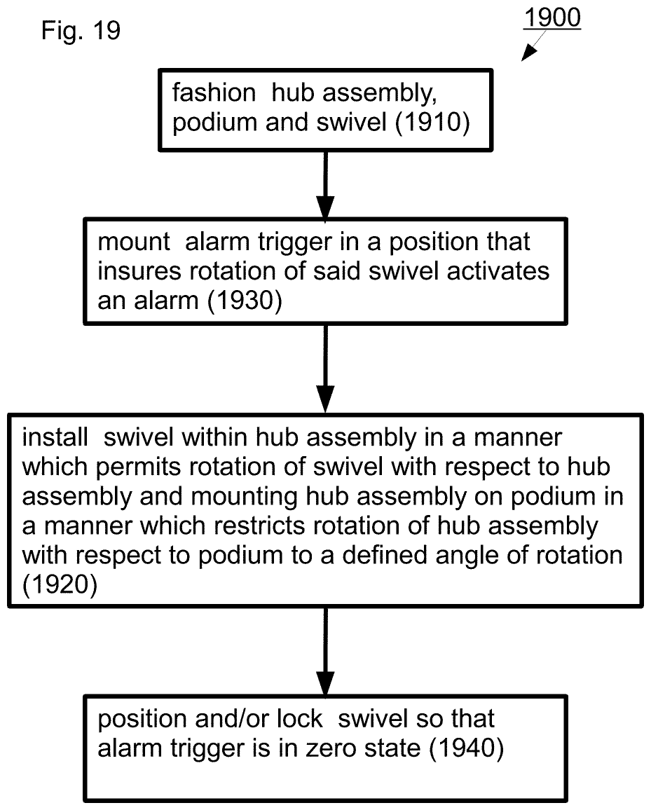

FIG. 19 is a simplified flow diagram of a method for manufacturing apparatus according to additional exemplary embodiments of the invention indicated generally as 1900.

Depicted exemplary method 1900 includes fashioning 1910 a hub assembly, a podium and a swivel and installing 1920 the swivel within the hub assembly in a manner which permits rotation of the swivel with respect to the hub assembly and mounting the hub assembly on the podium in a manner which restricts rotation of the hub assembly with respect to the podium to a defined angle of rotation. In the depicted embodiment, method 1900 includes mounting 1930 an alarm trigger in a position that insures rotation of the swivel activates an alarm. In some embodiments method 1900 includes positioning and/or locking 1940 the swivel so that the blade is in a desired starting orientation (zero state).

In some exemplary embodiments of the invention, when the alarm is triggered, the apparatus locks the catheter in place to prevent further linear travel with respect to the hub assembly.

In some exemplary embodiments of the invention, fashioning 1910 includes injection molding and/or co-injection and/or insert injection and/or over molding. Alternatively or additionally, in some embodiments fashioning 1910 includes additive manufacturing.

Alternatively or additionally, in some embodiments installing 1920 includes using connectors (e.g. screws, snaps, rivets, adhesive or glue).

In some embodiments mounting 1930 (of the alarm trigger as part of an assembled alarm mechanism) occurs prior to installing 1920 (swivel within the hub).

Exemplary Materials

According to various exemplary embodiments of the invention the hub and/or the swivel and/or the podium is constructed of metal and/or polymeric plastics and/or ceramic materials. In some exemplary embodiments of the invention, the hub, the swivel and the podium are each constructed of polymeric plastic and the blade is constructed of metal and/or ceramic material. In some embodiments the blade is constructed of metal.

Suitable metals include, but are not limited to steel (e.g. stainless steel), aluminum and aluminum alloys.

Suitable polymeric plastics include, but are not limited to thermoplastic materials including but not limited to ABS (Acrylonitrile-Butadiene-Styrene (Terpolymer)), PC-ABS (Polycarbonate/Acrylonitrile Butadiene Styrene), PU (polyurethane), PE (polyethylene), PP (polypropylene), PETG (Polyethylene Terephthalate Glycol), PET (Polyethylene Terephthalate), PC (polycarbonate), PA (polyamide), PS (polystyrene), PVC (polyvinylchloride) and POM (polyoxymethylene). According to various exemplary embodiments of the invention the hub assembly and/or swivel and/or podium are each independently constructed of one or more of these plastics.

According to various exemplary embodiments of the invention the blade is constructed of materials including, but not limited to Stainless steel and/or Razor Blade steel and/or Carbon Steel and/or Chrome Steel.

Exemplary Technical Specification

In some embodiments, apparatus according to various configurations described hereinabove have a total weight of about 50 g, about 40 g, about 30 g, about 20, about 10 g, about 5 g, about 2.5 g or intermediate or lesser weights. In some embodiments, a reduction in weight contributes to a reduction in the probability of patient discomfort or other adverse reaction.

In some embodiments, apparatus according to various configurations described hereinabove have overall dimensions of 40 mm width×35 mm height×13 mm thickness or less (with cover closed, excluding the pad).

In some embodiments, apparatus according to various configurations described hereinabove have overall dimensions of 50 mm width×40 mm height×20 mm thickness or less (with cover closed, excluding the pad).

In some embodiments, apparatus according to various configurations described hereinabove do not affect rate of flow of urine between the bladder and urine collecting bag by more than about 2%. Rate of flow is typically about 2.22 cc/sec on 16 Fr lumen catheters with normal kidney function.

In some embodiments, apparatus according to various configurations described hereinabove operate in an “all or nothing” fashion (i.e. there is no partial cutting).

Exemplary Use considerations

Although various embodiments of the apparatus described above are different in design, they are all easily installed by anyone that is accustomed to installing catheters or medical tubing. Training, if any is needed, should take only a few minutes. In many cases a single demonstration is enough. In other cases, no training is needed and the apparatus comes with installation instructions. In some embodiments, these instructions include a series of line drawings or photographs.

In some embodiments, the apparatus is removable by one person and without causing damage to the catheter or the patient (i.e. pulling the apparatus while catheter is being installed). Alternatively or additionally, in some embodiments the apparatus is removable without tools.

Alternatively or additionally, in some embodiments the apparatus does not ‘travel’ over the catheter length but can be manually adjusted to a desired point along the catheter length during placement.

In some embodiments, the apparatus has a place for writing time/date of installation and/or installer name.

According to various exemplary embodiments of the invention a length of tubing 99 accommodated by the channel (e.g. 219 in FIG. 2; 650 in FIG. 6; or in FIG. 13 the channel disposed between an outer surface 1332 of swivel 1330 and an inner wall 1312 of hub assembly 1310) is about 20 mm, 30, mm, 40 mm, 50 mm, 60 mm, 70, mm, 80 mm, 90 mm or 100 mm or intermediate or greater lengths. According to various exemplary embodiments of the invention a length of tubing 99 accommodated by the channel varies according to catheter type and/or catheter diameter and/or catheter material. Alternatively or additionally, an amount of linear travel required for cutting and/or or for triggering the alarm is about 10 mm, 20 mm or 30 mm or intermediate or greater amounts of linear travel. In some exemplary embodiments of the invention, the amount of linear travel required for cutting and/or for triggering the alarm is about 20 mm. According to various exemplary embodiments of the invention the amount of linear travel that causes cutting and/or alarm activation varies according to catheter type and/or catheter diameter and/or catheter material. It is noted that in some embodiments the defined angle of rotation of the hub assembly with respect to the podium provides a “buffer” of linear travel which can be used repeated without causing linear travel of the tubing with respect to the swivel.

It is expected that during the life of this patent many new materials and manufacturing processes will be developed and the scope of the invention is intended to include all such new technologies a priori.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Specifically, a variety of numerical indicators have been utilized. It should be understood that these numerical indicators could vary even further based upon a variety of engineering principles, materials, intended use and designs incorporated into the various embodiments of the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize an apparatus and features used to describe an apparatus can be used to characterize a method.

It should be further understood that the individual features described hereinabove can be combined in all possible combinations and sub-combinations to produce additional embodiments of the invention. The examples given above are exemplary in nature and are not intended to limit the scope of the invention which is defined solely by the following claims.

Each recitation of an embodiment of the invention that includes a specific feature, part, component, module or process is an explicit statement that additional embodiments of the invention not including the recited feature, part, component, module or process exist.

Alternatively or additionally, various exemplary embodiments of the invention exclude any specific feature, part, component, module, process or element which is not specifically disclosed herein.

Specifically, the invention has been described in the context of indwelling urinary catheters but might also be used in the context of other catheter types or medical tubing, wires or cables in general.

All publications, references, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “include”, and “have” and their conjugates as used herein mean “including but not necessarily limited to”.

1. An apparatus comprising:
(a) a hub assembly engaged by a podium in a manner which restricts rotation of said hub assembly with respect to said podium to a defined angle of rotation;
(b) a swivel installed within said hub assembly in a manner which permits rotation of said swivel with respect to said hub assembly; and
(c) a blade installed on an outer surface of said swivel, a cutting edge of said blade facing an inner wall of said hub assembly.

2. An apparatus according to claim 1, comprising a channel sized to engage and retain a catheter along a portion of its length, said channel disposed between an outer surface of said swivel and said inner wall of said hub assembly.

3. An apparatus according to claim 1, comprising protrusions on said outer surface of said swivel.

4. An apparatus according to claim 1, comprising an alarm trigger responsive to rotational motion of said swivel with respect to said hub assembly.

5. An apparatus according to claim 1, comprising a cover closeable over said swivel and said hub assembly.

6. An apparatus according to claim 4, comprising alarm circuitry responsive to said alarm trigger.

7. An apparatus according to claim 5, comprising a hinge connecting said cover to said hub assembly.

8. An apparatus according to claim 1, comprising a cover covering said swivel.

9. An apparatus according to claim 1, wherein said defined angle of rotation is in the range of 0° to 180° degrees.

10-19. (canceled)

20. A method comprising:
(a) inserting tubing into a patient;
(b) engaging a portion of said tubing in an apparatus with a rotating swivel and a cutting blade mounted thereon; and
(c) fixing said apparatus in place so that linear motion of said tubing causes rotation of said swivel and causes said cutting blade to cut said tubing.

21. A method according to claim 20, wherein said tubing is made of latex.

22. A method according to claim 20, wherein said tubing is made of silicon.

23-27. (canceled)

28. A method comprising:
(a) fashioning a hub assembly, a podium and a swivel;
(b) mounting a blade on an outer surface of said swivel, a cutting edge of said blade facing an inner wall of said hub assembly; and
(c) installing said swivel within said hub assembly in a manner which permits rotation of said swivel with respect to said hub assembly and mounting said hub assembly on said podium in a manner which restricts rotation of said hub assembly with respect to said podium to a defined angle of rotation.

29. A method according to claim 28, wherein said fashioning includes at least one process selected from the group consisting of injection molding, co-injection, insert injection and over molding.

30. A method according to claim 29, wherein said fashioning includes additive manufacturing.

31. A method according to claim 28, wherein said installing comprises using connectors.

32. A method according to claim 28, wherein said mounting comprises at least one process selected from the group consisting of insertion in a grove or slot, riveting and heat welding.

33- 37. (canceled)
